(19) United States
(12) Patent Application Publication
(10) Pub. No.: US 2013/0316993 A1
(43) Pub. Date: Nov. 28, 2013

(54) TRIGLYCERIDE COMPOSITIONS OF PLANT ORIGIN FOR COSMETIC APPLICATIONS OBTAINED FROM OLIVE OIL DEODORIZATION DISTILLATE

(71) Applicants: Giacomo Santus, Agrate Brianza (MB) (IT); Danilo Santoro, Agrate Brianza (MB) (IT); Adriano Busetti, Agrate Brianza (MB) (IT)

(72) Inventors: Giacomo Santus, Agrate Brianza (MB) (IT); Danilo Santoro, Agrate Brianza (MB) (IT); Adriano Busetti, Agrate Brianza (MB) (IT)

(73) Assignee: BIOPHIL ITALIA S.P.A., Agrate Brianza (MB) (IT)

(21) Appl. No.: 13/983,424

(22) PCT Filed: Mar. 29, 2012
(86) PCT No.: PCT/IB2012/051526

§ 371 Date: Aug. 2, 2013

(30) Foreign Application Priority Data
Mar. 29, 2011 (IT) MI2011A000507

(51) Int. Cl. A61K 8/92; A61Q 19/00; A61K 8/67; A61K 8/31; A61K 8/63

(52) U.S. Cl. CPC A61K 8/922; A61K 8/31; A61K 8/63; A61K 8/678; A61Q 19/00
USPC 514/171

(57) ABSTRACT

The present invention refers to a mixture of vegetable triglycerides containing saturated fatty acids with chain length C16-C18. The invention further relates to their composition, possibly in combination with other vegetable glycerides, to the techno logical process for their preparation, to their use for cosmetic applications.

### SUMMARY

The present invention refers to a mixture of vegetable triglycerides containing saturated fatty acids with chain length C16-C18. The invention further relates to their composition, possibly in combination with other vegetable glycerides, to the technological process for their preparation, to their use for cosmetic applications.

### DESCRIPTION

The present invention refers to a mixture of oils of vegetable origin, with triglycerides having fully saturated C16-C18 fatty acid chain (deposited INCI name “PALMITIC/STEARIC TRIGLYCERIDES”, hereinafter referred to as “C16-C18 triglycerides”), obtained by selection of specific non-edible heavy fractions deriving from the processing of the vegetable oils industry, especially of olive oil, and their use for cosmetic applications, particularly as thickeners of the lipophilic phases in emulsions.

The present invention further relates to combinations of C16-C18 triglycerides with other glycerides resulting from the vegetable oils industry, especially of olive oil, and their cosmetic applications.

A further object of the present invention are cosmetic formulations containing mixtures and compositions described above as thickeners for the lipophilic phase, with low environmental impact.

### BACKGROUND OF THE INVENTION

Vegetable triglycerides—also commonly referred to as vegetable oils—are very important natural products for cosmetic formulations, and for products for topical application in general. In nature a wide variety of such materials are available, potentially suitable for the preparation of cosmetic products. Vegetable triglycerides are natural esters composed by glycerol (glycerin) and long chain fatty acids. Their composition, and consequently their properties for cosmetic use, depends on the vegetable source, i.e. on the seed, fruit or tree which the oil is extracted from, and by the extraction process and the possible processing of the oil itself, for example for the food industry.

The primary composition of vegetable triglycerides largely depend on the chemical formula of fatty acids, especially on the length of the chain of carbon atoms, and on the number of unsaturations in the chain itself. Some oils—e.g. coconut and palm—are typically rich in saturated fatty acids, while others—e.g. olive oil—are rich in monounsaturated fatty acids. Typically, the melting temperature of triglycerides increases with increasing chain length of their fatty acids, and with decreasing index of unsaturation (i.e., at equal number of carbon atoms, a triglyceride with saturated chains melts at higher temperature of one with unsaturated chains).

The characteristics of the vegetable oils also depend on the concentration and composition of the so-called unsaponifiables fraction, i.e. the fraction which—not being composed of esters—does not undergo the process of saponification, known to the skilled person. The composition of this fraction again varies depending on the nature of the specific plant species, on the part which the oil is extracted from, on the extraction process and the following transformation processes.

In the modern cosmetics, vegetable triglycerides are used as emollients, thickeners, skin protectors, nourishing agents and moisturizers. An important feature for cosmetic use is that they must be as much as possible colorless and odorless, and stable to oxidation/yellowing; such a characteristic is typical of saturated vegetable triglycerides, as compared to unsaturated ones.

In particular, saturated vegetable triglycerides are mainly used in cosmetics as thickeners and stabilizers. The most common are (fractions of) palm, coconut, sunflower, corn, macadamia, sesame, sweet almond, castor, jojoba oils.

In cosmetics, thickeners—also known, depending on the specific use, as gelling agents, thickeners, consistency factors, rheology modifiers—are used to impart increased viscosity to the preparation, e.g. emulsions, massage oils, etc, in this way also promoting a greater stability of the emulsion. They are divided into thickeners for hydrophilic phases, and for lipophilic phases. The most common thickening agents for lipophilic phases for cosmetic applications are synthetic polymers (e.g. polybutenes, polypropylenes), synthetic esters (e.g. stearates, trimetyilsilico-oxysilicates, dimeticonol-behenate), silicone waxes, hydrogenated vegetable esters (e.g. hydrogenated/ethoxylated castor oil).

The unsaponifiable fraction of certain oils, particularly that of olive oil, has known properties, e.g. antioxidant and protective, for cosmetic applications. In Olea Europaea (hereinafter simply referred to as olive oil) the unsaponifiable fraction amounts to approx. 1-1,5% of the oil, while the remaining saponifiable fraction is composed mostly of triglycerides, with a minor component of di- and mono-glycerides (ie esters of glycerol with respectively only 2 or 1 hydroxyl groups esterified with fatty acids).

In virgin olive oil the components of the unsaponifiable fraction are hydrocarbons (mainly terpenes, among them predominantly squalene), waxes (present in small quantities in the extra-virgin oil, and in greater quantities in olive pomace), triterpenic alcohols, sterols (mostly beta-sitosterol), pigments (mainly carotenoids and chlorophyll), hydro-and lipo-soluble antioxidants (polyphenols and tocopherols). They have a recognized dermocosmetic activity, due to e.g. the soothing and protective properties of squalene, and the anti-ageing protection by the lipophilic and hydrophilic antioxidant components, and they enjoy wide commercial use.

### STATE OF THE ART

In WO2009/056275 (Santus et al.) a mixture is described, mainly composed of di- and tri-glycerides, preferably with 25:75 ratio, and in which the fraction of unsaponifiables is not present, obtained by successive steps of purification of olive oil. The composition of fatty acids is typically the same as contained in olive oil. The described mixture has cosmetic applications as emulsifier and carrier of active ingredients.

In EP 1018540 A1, a mixture of unsaponifiables and triglycerides from distillates of deodorization is achieved through a process of purification in subsequent steps, including an esterification with mono-alcohols, to obtain a final mixture with high content in unsaponifiables, and also containing monoesters of C16-C18 saturated acids.

In WO 02/26 207 A2, a mixture of unsaponifiables from olive oil, and from other raw vegetable oils of different origins, is described. The mixture has the appearance of a viscous oil.

In GB 884 688 A, a mixture for cosmetic use is described, rich in triglycerides of saturated and unsaturated fatty acids, waxes, and squalene, of vegetable and animal origin.

In WO 02/50 221 A1, a mixture is described, enriched in unsaponifiables (predominantly phytosterols and triterpenes), obtained through a process of purification in subsequent steps of raw oils or butters, including an initial step of hydrogenation. The final mixture there described also contains saturated and unsaturated triglycerides (the latter prevailing).

### DESCRIPTION OF THE INVENTION

Object of the invention is a composition for cosmetic use, containing a mixture of triglycerides enriched in fatty acid esters with saturated C16-C18 chain, and an unsaponifiable fraction, obtained by the selection of specific non-edible heavy fractions from the processing of the vegetable oils industry, particularly of olive oil. Compared to other esters having the same function as a thickener in cosmetic formulations, the mixture has the advantage of being of vegetable origin and of having a positive environmental impact assessment according to the method of Life Cycle Assessment (LCA). Another advantageous aspect of the C16-C18 triglycerides according to this invention, when compared to esters obtained by synthetic or semi-synthetic routes, and/ or obtained by hydrogenation of unsaturated esters, is the presence of a residual heavy fraction of unsaponifiables, exerting recognized cosmetic activity, e.g. as emollients and antioxidants.

According to the invention, the mixture composed of triglycerides enriched in fatty acid esters with saturated C16-C18 chain and the unsaponifiable fraction is obtained from the deodorization distillates through the following procedure:

1) the distillate is dispersed in cold water through the use of a surfactant and an electrolyte (adjuvants of crystallization of the fraction of saturated fatty acids);

2) after being left a few hours at ambient temperature (“maturazione”), the dispersion is subjected to rapid “retempering”, then separated by centrifugation, obtaining a solid fraction, rich in free palmitic and stearic fatty acids, and a liquid fraction, consisting mainly of oleic and linoleic acids, and the entire fraction of unsaponifiables (represented by approx. 70% squalene; this process, and the conditions for the dissolution, surfactants, electrolytes are known to the skilled person with the name of “wet fractionation”);

3) the solid fraction, rich in C16-C18 saturated acids—with a palmitic/stearic acid ratio of approx. 3.5-6:1—is then subjected to esterification by addition of glycerol of vegetable origin, preferably obtained by purification of olive oil, in excess of 10% compared to the stoichiometric ratio; the operation is carried out under vacuum, heating from 165° C. up to 230° C. under forced circulation, and monitoring the production of water and the residual acidity; the esterification is considered complete when there is no more production of reaction water, and the free acidity reaches a value of approx. 2-3%;

4) the excess acidity is then neutralized by adding caustic soda (12% vol.) at 90° C., the soapy mixture is washed—preferably 2 times—with demineralized water, and the mixture is dried under vacuum;

5) optionally the fraction is further subjected to bleaching and deodorization according to techniques well-known to the skilled person; in this way a product is obtained, containing mainly palmitic/stearic triglycerides in a ratio of approx. 3.5-6:1, which is the same found in virgin olive oil, with pale color, no residual odor, and low acidity;

and, independently from steps 3-5

6) the liquid fraction referred to in point 2, rich in unsaturated triglycerides (mainly oleic acid and linoleic acid) and unsaponifiables, is subjected to the process of saponification with potassium hydroxide, known to the skilled person, to eliminate the triglyceride fraction;

7) optionally, the fraction of unsaponifiables, consisting predominantly of squalene (approx. 70%), sterols and tocopherols can be further purified by distillation in high vacuum (1 mbar residual pressure) from 100 to 250° C.;

8) the fraction referred to in points 6 or 7 is then joined to the product referred to in points 5 or 6.

The starting deodorization distillate may be obtained by refining virgin olive oil (“lampante”) by the following sequence, well known to the skilled person:
degumming (“degommazione”) with phosphoric acid and washing
continuous neutralization with alkali
separation of soaps by centrifugation, washing, and continuous drying of the obtained neutral oil
discoloration on vegetable earths or vegetable activated carbons, and filtration on horizontal press filter or vertical self-cleaning filters
deodorization in high vacuum at 1-3 mbar of residual pressure and 180-220° C. in deodorization column.

The deodorization distillate obtained according to the above process has a composition in free acids which is very similar to that of starting crude olive oil, and a content of unsaponifiables of approx. 10-15% of the total. Of the unsaponifiable fraction, approximately 70% consists of squalene, along with a good presence of (free) sterols and tocopherols.

In one mode of the invention, together with the deodorization distillates of refining crude olive oil (“lampante”), other residual heavy fractions of the vegetable oil processing of other species, e.g. palm, corn, sunflower, etc., are used as a starting material.

In another mode of the invention, for an easier use by the cosmetic formulator, a greater safety for the operator and the work environment, and for a reduction of wastes, the C16-C18 triglycerides are preferably further processed in the form of beads, called “pearls”, according to the following steps:

1) the fractions from previous points, solid at room temperature, are heated up to a temperature of 90° C. in a melting vessel equipped with mechanical stirring;

2) after the mixture is completely melted, the temperature is lowered to 80-85° C.;

3) from the melting vessel the product is cast through a tap, on a percolator with rotating toothed shaft, and from here it is dripped on a refrigerant rotating band of a length of 18-20 m and at temperature of 8° C.;

4) at the end of the rotating band, the thus obtained solidified pearls are collected in the desired packaging.

A further aspect of the invention concerns the combination of the composition described above with other glycerides deriving from the vegetable oil industry, especially olive oil, and their use for cosmetic applications.

In one embodiment of this invention, the composition is formulated together with a fraction of olive glycerides consisting of a mixture of tri- and di-glycerides in 75:25 typical ratio (this fraction, in the specific ratio of 75: 25, will hereinafter be called “Olive glycerides”, INCI name “Olive Glycerides”). This fraction shows some peculiarities in cosmetic use, such as the ability to modulate the lipophilic/hydrophilic balance, to impart a silky non-greasy touch and to act as carrier of hydrophilic active ingredients into the dermis, and being completely odorless and colorless. In a preferred embodiment, the Olive Glycerides are prepared as described in WO2009/056275, the content of which is incorporated herein by reference.

The formulation containing Olive glycerides is obtained by applying the following steps:

1. in a standard turbo-emulsifier, the Olive glycerides are added and the turboemulsifier is heated up to 70° C., with continuous stirring at slow pace;

2. when the temperature is reached, the mixture of C16-C18 triglycerides is added in one portion, stirring until complete dissolution;

3. after having obtained a clear transparent solution, the material is cooled with continuous slow stirring; below 50° C. a homogeneous paste is obtained;

4. upon reaching 45° C., the turbine of the turboemulsifier is triggered for few minutes, until a completely homogeneous paste is obtained;

5. samples for quality control are then taken and any adjustments are made with addition of one of the two materials, to obtain the desired chemical-physical parameters;

6. the material is allowed to cool down to 40° C., and then loaded in special containers with wide opening;

7. the containers are stored at temperatures between 20-25° C.

The thus obtained product shows the form of a butter, white and practically odorless in solid form, and colorless with slight pleasant odor in molten form.

The C16-C18 triglycerides of the Olive glycerides may be blended to achieve a butter solid at 23° C., up to a minimum ratio C16-C18 triglycerides/

Olive glycerides of 1:4. At higher concentration, the Olive glycerides tend to separate at the surface. In a preferred embodiment, this ratio is exactly 1:4. Unexpectedly, at this ratio a butter is obtained, with optimal consistency at 23° C., but that it melts quickly at body temperature. The butter thus obtained is very useful for the cosmetic formulator, because:
it is a great consistency factor and gelling agent for the lipophilic phase of cosmetic emulsions (skin care, body-care, sun-care) at room temperature and up to 30° C.
by varying the percentage of the material in the finished cosmetic formulation, it allows to modulate the lipophilic/hydrophilic balance, functioning as emulsifier
in contact with the body surface during the spreading of the finished cosmetic formulation, it melts improving the spreadability and leaving a pleasant emollient effect and a silky touch
it can be used as a vector of hydrophilic active ingredients to the dermis.

In a further embodiment of the invention, the mixture of C16-C18 triglycerides is formulated with olive oil, preferably extra-virgin olive oil, still more preferably one obtained by mere mechanical pressing (most preferred is the oil of Italian origin, and Tuscany in particular).

The formulation of C16-C18 triglycerides with extra-virgin olive oil is obtained by applying the following steps:

1. in a standard turbo-emulsifier, the extra-virgin olive oil is added and the turbo-emulsifier is heated up to 70° C. with continuous stirring at slow pace;

2. when the temperature is reached, the mixture of C16-C18 triglycerides is added in one portion, stirring until complete dissolution;

3. after having obtained a clear transparent solution, the material is cooled down with continuous slow agitation; below 50° C. a homogeneous paste is obtained;

4. upon reaching 45° C., the turbine of the turboemulsifier is triggered for few minutes, until a completely homogeneous paste is obtained;

5. are then taken of the samples for quality control and any adjustments made with additions of one of the two materials, to obtain the desired chemical-physical parameters.

6. the material is allowed to cool down to 40° C., and then loaded in special containers with wide opening;

7. the containers are stored at temperatures between 20-25° C. The product obtained is again in the form of a butter, with a typical light green color and almost odorless in solid form, and with a tenuous color and pleasant odor, typical of extra-virgin olive oil in molten form.

The C16-C18 triglycerides and the extra-virgin olive oil can be blended to achieve a solid butter at 23° C. in a minimum ratio of C16-C18 triglycerides/extra-virgin olive oil of 3:7.

A further object of the present invention are cosmetic formulations obtained with the products referred to above. These formulations are produced preferably in the form of creams, gels, ointments, cosmetic oils, massage oils, body/hands/face lotions, water/oil or oil/water emulsions, detergents, shampoos, hair lotions, bath gels and foams, shower gels and foams, liquid soaps, sticks, make-up products.

By way of example some formulations are reported here below, containing C16-C18 triglycerides in pearls, C16-C18 triglycerides+Olive glycerides in butter, C16-C18 triglycerides+extra-virgin olive oil in butter.

The C16-C18 triglycerides are reported with their deposited INCI denomination “PALMITIC/STEARIC TRIGLYCERIDE”.

For demonstration, the gelling/thickening effect of C16-C18 triglycerides has been tested with some components which are typically used in the oil phase of a cosmetic formulation (vegetable, mineral and synthetic oils), and with some typical cosmetic formulations.

FIGS. 1-5 show the thickening effects of C16-C18 triglycerides at different percentages of use (2.5-5-10-15%) with the major oils used in cosmetics. It may be noticed a high affinity with vegetable oils, a good affinity with silicones and esters, and a poor affinity with short chain mineral oils.

It may be noted that the synergistic effect is greater with vegetable oils, which have a more similar chemical structure to that of C16-C18 triglycerides. It should also be noted that the greater the initial viscosity of the oil and its freezing point, and the greater the thickening effect of C16-C18triglycerides; this observation also explains the poor affinity with short chain mineral oils.

The synergistic effect also depends on the % of C16-C18 triglycerides, with specific characteristics which vary depending on the starting oil, and which usually show an optimum around 10-15%, as is clear from FIG. 5.

The thickening and stabilizing effect of C16-C18 triglycerides is shown by way of example here below in the formulations of Examples 1-3 (values of relative viscosity in cps at room temperature, without and with addition of 2.5% of C16 C18 triglycerides):
Hand cream Example 1. Without: 75,600 cPs; with 2.5%: 138,600 cPs; +83% increase in viscosity;
Body cream Example 2. Without: 27,200 cPs, with 2.5%: 54,000 cPs; +99% increase in viscosity;
Face Cream Example 3. Without: 226,200 cPs, with 2.5%: 276,300 cPs; +22% increase in viscosity.

This effect allows the formulator to use lower percentages of other waxes and emulsifiers in cosmetic compositions.

### Example 1

### “HAND CREAM”, Standard Hand Cream with and without C16-C18 Triglycerides

| Commercial | | |
|---|---|---|
| Name | INCI Denomination | % |
| A | | |
| | AQUA | q.b. |
| | DISODIUM EDTA | 0.1 |
| | PANTHENOL | 1 |
| | GLYCERIN | 1 |
| B | | |
| | CETYL ALCOHOL (AND) CETEARETH-20 | 8 |
| | (AND) STARYL ALCOHOL | |
| | ISOPROPYL PALMITATE | 4 |
| | OCTYLDODECANOL | 2 |
| | PALMITIC/STEARIC TRIGLYCERIDE | 0 o 2.5 |
| | CETEARYL ALCOHOL | 4 |
| | PRUNUS AMYGDALUS DULCIS (SWEET | 0.2 |
| | ALMOUND) OIL | |
| | TOCOPHERYL ACETATE | 0.2 |
| C | | |
| ISOCIDE C | METHYL CHLOROISOTHIAZOLINONE, | 0.05 |
| | METHYLISOTHIAZOLINONE, | |
| | MAGNESIUM NITRATE, MAGNESIUM | |
| | CHLORIDE | |
| D | | |
| | CYCOLPENTASILOXANE (AND) | 5 |
| | DIMETHICONOL | |
| | PARFUM | 0.2 |

### Example 2

### “BODY CREAM”, Standard Firming Body Cream with and without C16-C18 Triglycerides

| Commercial | | |
|---|---|---|
| Name | INCI Denomination | % |
| A | | |
| | CETEARYL ALCOHOL (AND) CETEARYL | 3.00 |
| | GLUCOSIDE | |
| | GLYCERYL STEARATE | 1.50 |
| | OCTYLDODECANOL | 5.00 |
| | PALMITIC/STEARIC TRIGLYCERIDE | 0 o 2.5 |
| | CETYL PALMITATE | 2.00 |
| | CETEARYL ETHYLHEXANOATE | 8.00 |
| GEMSEAL 70 | C18-21 ALKANE | 5.00 |
| B | | |
| | AQUA | q.b. |
| POLACRIL 41 | CARBOMER | 0.10 |
| | DISODIUM EDTA | 0.10 |
| | UREA | 1.00 |
| | GLYCERIN | 1.50 |
| C | | |
| | AQUA | 5.00 |
| | IMIDAZOLIDINYL UREA | 0.30 |
| | PHENOXYETHANOL (AND) | 0.80 |
| | METHYLPARABEN (AND) | |
| | BUTYLPARABEN (AND) | |
| | ETHYLPARABEN (AND) | |
| | PROPYLPARABEN | |
| | FRAGRANCE | 0.2 |

### Example 3

### “FACE CREAM”, Standard Face Cream with and without C16-C18 Triglycerides+Olive Glycerides

| Commercial | | |
|---|---|---|
| Name | INCI Denomination | % |
| A | | |
| | CETEARYL ALCOHOL | 3.35 |
| | ARACHIDYL BEHENYL ALCOHOL | 3.75 |
| | (AND) ARACHIDYL GLUCOSIDE | |
| | CETEARYL ALOCHOL (AND) | 3.75 |
| | CETEARYL GLUCOSIDE | |
| | OLIVE GLYCERIDES (AND) | 0 o 3.75 |
| | PALMITIC/STEARIC TRIGLYCERIDE | |
| | HYDROGENATED POLYDECENE | 37.50 |
| B | | |
| | AQUA | qb |
| | GLYCERIN | 5.00 |
| | DISODIUM EDTA | 0.25 |
| | PANTHENOL | 2.50 |
| C | | |
| ISOCIDE C | METHYL CHLOROISOTHIAZOLINONE, | 0.05 |
| | METHYLISOTHIAZOLINONE, | |
| | MAGNESIUM NITRATE, MAGNESIUM | |
| | CHLORIDE | |
| | FRAGRANCE | 0.625 |

### Example 4

### “DAY CREAM WITH SPF”, Day Cream with Solar Protection Factor Containing C16-C18 Triglycerides

| Commercial | | |
|---|---|---|
| Name | INCI Denomination | % |
| | ISOCETETH-10 STEARATE (E) | 3.00 |
| | ISOSTEARETH-10 STEARATE | |
| | ACETYLATED GLYCOL STEARATE | 2.00 |
| | CETEARYL ALCOHOL | 3.00 |
| | PALMITIC/STEARIC TRIGLYCERIDE | 3.00 |
| GEMSEAL 40 | C17-21 ALKANE | 2.00 |
| | TITANIUM DIOXIDE | 3.00 |
| | ZINC OXIDE | 1.00 |
| | OCTOCRYLENE | 2.00 |
| | BUTYL | 1.00 |
| | METHOXYDIBENZOYLMETHANE | |
| | TOCOPHERYL ACETATE | 0.70 |
| | WATER | Q.B. |
| | | a 100 |
| POLACRIL 40 | CARBOMER | 0.20 |
| | GLYCERIN | 2.00 |
| | DISODIUM EDTA | 0.10 |
| ISOCIDE C | METHYL CHLOROISOTHIAZOLINONE, | 0.05 |
| | METHYLISOTHIAZOLINONE, | |
| | MAGNESIUM NITRATE, MAGNESIUM | |
| | CHLORIDE | |
| | TRIETHANOLAMINE | Q.B. a pH |
| TOTAL | | 100.00 |

### Example 5

### “BODY EMULSION”, Fluid Body Cream, Containing C16-C18 Triglycerides

| Commercial | | |
|---|---|---|
| Name | INCI Denomination | % |
| A | | |
| | OLUS OIL, GLYCERIN, LAURYL | 6.50 |
| | GLUCOSIDE, POLYGLYCERYL-2 | |
| | DIPOLYHYDROXYSTEARATE, | |
| | GLYCERYL OLEATE, DICAPRYLYL | |
| | CARBONATE | |
| | CETEARYL ALCOHOL | 1.50 |
| | PALMITIC/STEARIC TRIGLYCERIDE | 2.00 |
| | OCTYLDODECANOL | 5.00 |
| | TOCOPHERYL ACETATE | 0.50 |
| NIPSEAL PP4 | C21-28 ALKANE, HYDRATED SILICA, | 5.00 |
| | MAGNESIUM SILICATE | |
| B | | |
| | AQUA | 78.15 |
| | DISODIUM EDTA | 0.10 |
| | GLICERYN | 1.00 |
| POLACRIL 40 | CARBOMER | 0.20 |
| C | | |
| | TRIETHANOLAMINE | Q.B. |
| ISOCIDE LINE | PRESERVATIVES | 0.05 |
| | | 100.00 |

### Example 6

### “BUTTER ENZYME PEELING”, Butter Scrub with Enzymes, Containing C16-C18 Triglycerides+Olive Glycerides

| Commercial | | |
|---|---|---|
| Name | INCI Denomination | % |
| A | | |
| NIPSEAL PP4 | C21-28 ALKANE, HYDRATED | 16.30 |
| | SILICA, MAGNESIUM | |
| | SILICATE | |
| ABWAX WHITE | ALBA WAX | 8.00 |
| BEESWAX PEARLS | | |
| ABWAX CANDELILLA | CANDELILLA WAX | 4.00 |
| LUMPS PEARLS | | |
| ABWAX | MICROCRYSTALLINE WAX | 3.00 |
| MICROCRYSTALLINE | | |
| 171 PEARLS | | |
| B | | |
| | BEHENYL ALCOHOL, | 0.40 |
| | BUTYROSPERMUM PARKII, | |
| | HYDROGENATED CASTOR | |
| | OIL, STEARYL ALCOHOL, | |
| | PROTEASE, SUBTILISIN | |
| OLIFEEL TD7525 | OLIVE GLYCERIDES | 1.60 |
| C | | |
| | OLIVE GLYCERIDES (AND) | 3.00 |
| | PALMITIC/STEARIC | |
| | TRIGLYCERIDE | |
| GEMSEAL 120 | C15-19 ALKANE | 10.00 |
| OLIFEEL ORGANIC OIL | OLEA EUROPEA OLIVE OIL | 20.00 |
| | ETHYLHEXYL PALMITATE | 33.00 |
| | LECITHIN, TOCOPHEROL, | 0.20 |
| | ASCORBYL PALMITATE | |
| ISOCIDE LINE | PRESERVATIVES | q.b. |
| D | | |
| | PARFUM | 0.50 |
| | | 100.00 |

### Example 7

### “AFTER SUN MILK”, After-Sun Milk, Containing C16-C18 Triglycerides+Olive Glycerides

| Commercial | | |
|---|---|---|
| Name | INCI Denomination | % |
| A | | |
| | CETEARYL ALCOHOL, | 4.00 |
| | CETEARYL GLUCOSIDE | |
| | C12-15 ALKYL BENZOATE | 3.00 |
| OLIFEEL TD7525 | OLIVE GLYCERIDES | 2.00 |
| | ISONONYL ISONONANOATE | 2.00 |
| | OLIVE GLYCERIDES (AND) | 10.00 |
| | PALMITIC/STEARIC | |
| | TRIGLYCERIDE | |
| | VITAMIN E ACETATE | 0.10 |
| | MENTHOL | 0.05 |
| B | | |
| | AQUA | 74.95 |
| | GLYCERIN | 3.00 |
| | PROPYLENE GLYCOL | 0.10 |
| | XANTHAN GUM | 0.15 |
| | HYDROXYMETHYLCELLULOSE | 0.30 |
| | DISODIUM EDTA | 0.05 |
| | SODIUM HYALURONATE | 0.05 |
| C | | |
| ISOCIDE LINE | PRESERVATIVES | 0.05 |
| D | | |
| | PARFUM | 0.20 |
| | | 100.00 |

### Example 8

### “LIP BALM”, Lip Balm, Containing C16-C18 Triglycerides+Olive Glycerides

| Commercial | | |
|---|---|---|
| Name | INCI Denomination | % |
| A | | |
| | C16-18 TRIGLYCERIDES | 12.00 |
| | OCTYLDODECANOL | 36.00 |
| | OLIVE GLYCERIDES (AND) | 8.00 |
| | PALMITIC/STEARIC | |
| | TRIGLYCERIDE | |
| OLIFEEL TD 7525 | OLIVE GLYCERIDES | 43.30 |
| PHYLCARE | OLIVE GLYCERIDES, | 0.50 |
| CERAMIDE L | CERAMIDE III | |
| B | | |
| | PARFUM | 0.20 |
| | | 100.00 |

### Example 9

### “SOOTHING GEL FOR HEAVY LEGS”, Dense Gel with Soothing and Anti-Inflammatory Action, Containing C16-C18 Triglycerides+Olive Glycerides

| Commercial | | |
|---|---|---|
| Name | INCI Denomination | % |
| A | | |
| | AQUA | 79.30 |
| ISOCIDE LINE | PRESERVATIVES | 0.30 |
| POLACRIL 40 | CARBOMER | 1.50 |
| | MANNITOL, SODIUM CITRATE, | 0.30 |
| | ACETYL TETRAPEPTIDE-15 | |
| | AQUA, PROPYLENE GLYCOL, | 1.00 |
| | VACCINUM MYRTILLUM | |
| | EXTRACT | |
| | AQUA, PROPYLENE GLYCOL, | 1.00 |
| | VITIS VINIFERA EXTRACT | |
| | AQUA, PROPYLENE GLYCOL, | 1.00 |
| | CHAMOMILLA RECUTITA | |
| | EXTRACT | |
| B | | |
| | TRIETHANOLAMINE | 1.50 |
| C | | |
| DERMAPHYL | PEG-40 HYDROGENATED | 7.00 |
| SOLPLUS | CASTOR OIL, TRIDECETH-9, | |
| | POLYSORBATE-20 | |
| | OLIVE GLYCERIDES (AND) | 2.00 |
| | PALMITIC/STEARIC | |
| | TRIGLYCERIDE | |
| | MENTHOL | 2.50 |
| | PHENOXYETHANOL, | 2.50 |
| | GLYCERYL UNDECYLENATE, | |
| | BUTYLENE GLYCOL, | |
| | POLYAMINOPROPYL | |
| | BIGUANIDE | |
| D | | |
| | CI18050 | 0.10 |
| | | 100.00 |

### Example 10

### “BUTTER MASSAGE”, Massage Butter with Melting at Body Temperature, Containing C16-C18 Triglycerides+Olive Glycerides

| Commercial | | |
|---|---|---|
| Name | INCI Denomination | % |
| A | | |
| NIPSEAL PP4 | C21-28 ALKANE, HYDRATED | 13.30 |
| | SILICA, MAGNESIUM | |
| | SILICATE | |
| ABWAX WHITE | ALBA WAX | 8.00 |
| BEESWAX PEARLS | | |
| ABWAX CANDELILLA | CANDELILLA WAX | 4.00 |
| LUMPS PEARLS | | |
| ABWAX | MICROCRYSTALLINE WAX | 3.00 |
| MICROCRYSTALLINE | | |
| 171 PEARLS | | |
| | OLIVE GLYCERIDES (AND) | 6.00 |
| | PALMITIC/STEARIC | |
| | TRIGLYCERIDE | |
| ISOCIDE LINE | PRESERVATIVES | 0.80 |
| B | | |
| | BEHENYL ALCOHOL, | 0.40 |
| | BUTYROSPERMUM PARKII, | |
| | HYDROGENATED CASTOR | |
| | OIL, STEARYL ALCOHOL, | |
| | PROTEASE, SUBTILISIN | |
| OLIFEEL TD7525 | OLIVE GLYCERIDES | 1.60 |
| C | | |
| GEMSEAL 120 | C15-19 ALKANE | 10.00 |
| | JOJOBA OIL | 0.50 |
| | ETHYLHEXYL PALMITATE | 51.70 |
| | LECITHIN, TOCOPHEROL, | 0.20 |
| | ASCORBYL PALMITATE | |
| | PARFUM | 0.50 |
| | | 100.00 |

### Example 11

### Environmental Impact Assessment of C16-C18 Triglycerides in Real Formulations

A peculiar characteristic of C16-C18 triglycerides is to be derived from by-products of the vegetable oil food industry, and therefore to have a potentially positive effect on environmental impact, e.g. as compared to commonly used oils from palm or coconut. This hypothesis was then verified through an environmental impact analysis, conducted according to the approach of Life Cycle Assessment (LCA), using the SimaPro software, and the two methods ReCiPe and Ecoindicator 99. The analysis was carried out starting from the secondary data present in the software. Where some data were missing, materials have been selected, which data were considered the best approximation in the most proper manner the environmental impact under study. The collected primary data focused instead on some manufacturing technologies (transformation in beads (“pearls”), mixing, emulsification, treatment of formulation water, etc.).

The considered manufacturing process involves the following steps:
The purchase of bulk commodities, including by-products of the vegetable oil food industry, which are the starting raw material of C16-C18 triglycerides
The production of cosmetic ingredients and semi-finished products, including C16-C18 triglycerides in their end physical form (“pastigliazione”, i.e. transformation in beads)
Production of the cosmetic formulation bulk, through the use of a turbo-emulsifier, comprising the use of water treated with ion exchange resins and UV lamps, and bulk packaging (typically in drums of 25 kg)
Packaging and shipping of finished cosmetic at mass retailers

The evaluation of impact on C16-C18 triglycerides was carried out with the “from cradle to gate” approach, i.e. an analysis that starts from the extraction of raw materials and arrives to the finished product, provided with primary packaging, ready for the phase of distribution to the consumer. Within the study are, therefore, included extraction, transportation and processing of raw materials up to the finished cosmetics at the companies.

The evaluation was carried out on the formulation of Example 1 (“day cream with SPF”), with respect to a reference formulation. Compared to the reference formulation, in the “Day Cream with SPF” the C16-C18 triglycerides functionally replace a synthetic thickening agent typically used in the industry, the caprylic-capric triglyceride. Another change is given by the replacement of a silicon product, dimethicone, with alkanes derived from petrochemicals (alkanes C17-C21), more functional for the formulation of Example 1. All other components remain unchanged in the formulation.

The functional unit chosen for the analysis is equal to 1 kg of finished product (cream ready for distribution). The data in the inventory (LCI) were, for the most part, obtained from the libraries of the SimaPro software, in particular from Ecoinvent database and ETH-ESU 96; when data were recovered from those belonging to the Ecoinvent database, it was preferred to use the process units. Subject to the availability of information, it was however chosen to include all the processes considered in the portion of the analysis portion of the considered life cycle. Since there is now a rather limited number of studies concerning cosmetic products and their ingredients, in the construction of the LCI inventory, were identified among the substances contained in the available databases, those most suitable to represent the ingredients that compose the analyzed formulations. We chose to operate according to a principle of caution, choosing, under conditions of uncertainty, the least impacting alternative. The presented results therefore refer to the best possible hypothesis in terms of generated environmental impact.

The main assumptions made in this study are shown below:
Modeling materials and Cut-offs: non-present substances were modelled by substances, equivalent by molecular form or by production, refining and synthesis processes, present in the SimaPro database. In addition, only ingredients present in the formulations in a percentage equal to, or greater than 1%, were taken into account (cut-off).
Used equipment: as a comparative analysis, we used the same equipment for the process of emulsion (as a model we chose a turbo-emulsifier with capacity of 350 L), estimating the power consumption at partial loading of 300 L, mimicking the real conditions of a typical bulk cosmetics manufacturer in Northern Italy.
Sources of C16-C18 Triglycerides: It was decided to establish the origin of the vegetable oil industry by-products from Imperia, as a real case of e.g. olive oil.
Origin of Sodium Chloride: Given the uncertainty regarding the origins of sea salt sold in Italy (Puglia, Sardinia, Sicily, Northern Africa), a site of national production was chosen, the Region of Puglia, assuming that the product came from the salt production sit of Margherita di Savoia (BT).
Origin of Titanium dioxide, zinc oxide and glycerine: Because of the difficulties in finding information on the producers of titanium dioxide, zinc oxide and glycerol (the fraction of synthetic origin), a further web search was performed from which it was found that most of the production plants for these compounds are located in Germany. To simplify the system, it was therefore chosen to let these goods cross a stretch of road equal to that which leads from the company to Berlin.
Glycerine: Due to the uncertainties about the geographical provenience and its origin (natural or synthetic), we adopted a process built on 1 kg of glycerine, half of which was of natural origin (derived from palm oil, Malaysia, Kuantan port) and half was of synthetic origin, coming from Europe (Germany).
Road transportation: Where suitable, road transportation was preferred over other means, as the best mimicking option of real scenario. For most cases, it was assumed that this was done through the use of vehicles weighing approximately in the range of 16-32 tonnes, with diesel engines, Euro 3 class.
Overseas transportation: For all cases of trans-oceanic transportation, it was assumed that this is via cargo ship. Furthermore, as unloading port of ships coming from India and Malaysia, Bari port has been chosen; for those coming from the United States instead, the choice was made on New York as loading port and Genoa as unloading port.
Production facilities: Due to the relative difficulty in obtaining information concerning the actual location of production facilities of some suppliers of raw materials, a precautionary choice was made to consider the closest plant to the production facilities of the Italian cosmetics company.
Replacement of processes related to cosmetic ingredients not present in the database: If not present, the processes related to their production have been replaced by more viable alternatives present in the libraries of the software; the replaced processes:
Isoceteth-10-stearate and Isosteareth-10-stearate replaced by the process Vegetable oil methyl ester.
Acetylated Glycol Stearate acetylated replaced by Ethoxylated alcohols.
For the Caprylic/Capric Triglyceride the two processes Fatty alcohol from vegetable oil and Glycerine from vegetable oil were joined.
Dimethicone replaced by silicone or product.
Octocrylene and Butyl Methoxydibenzoylmethane replaced by Benzyl chloride.
C17-21 alkanes replaced by White Mineral Oil, petrochemical origin, modified process.

To properly model the production process of the C16-C18 triglycerides, two processes were used (glycerine and fatty acids, both of vegetable origin), which the raw materials have been removed from. The amount of raw material used to produce the C16-C18 triglycerides has been eliminated since thos come from processing by-products of another industry.

1. Results of the Evaluation with the ReCiPe Method

As is typical in a cosmetic emulsion, all the ingredients except water are present in relatively small amounts, which do not exceed 3%, in spite of this, some will generate a significant impact. On the opposite, formulation water does not generate a significant impact.

The Caprylic/Capric Triglyceride present at 3% in the reference formulation, shows a limited contribution, except for the category Agricultural land occupation, in which reaches a percentage of 48%, and to a lesser extent for the categories Natural land transformation and Terrestrial Ecotoxicity: this substance is derived, indeed, from the processing of vegetable oils.

The graph in FIG. 2 shows the environmental impact assessment with the Recipe method for Example 1 of the formulations, i.e. where—compared to the reference formulation—the caprylic/capric triglyceride and dimethicone (silicone oil) are replaced by C16-C18 triglycerides and C17-21 Alkanes.

Compared to the reference formulation, the impact of C16-C18 triglycerides is particularly low (max 3%), and is evenly distributed in all the analyzed categories. The fact that it derives from processing scraps of an existing production chain implies that the impacts related to the production of raw materials are not considered, and that its effect on Terrestrial Ecotoxicity, Agricoltural land occupation and Natural land transformation is in fact null.

The sum of the impacts generated in the formulation of Example 1 is less than 8% compared to the one of reference, and this reduction is due mostly to the C16-C18 triglycerides. The detailed comparison of all categories of impact are reported in Table 1: for the most part, the environmental performance of the formulation in Example 1 is positive.

The greatest benefits are found in Agricultural land occupation, which affects the use of C16-C18 triglycerides, and Ozone Depletion and Terrestrial Ecotoxicity, where the contributions of dimethicone and caprylic/capric triglyceride are absent right because they are replaced. The increase in Human toxicity and Fossil depletion categories can be attributed to the use of C17-21 alkanes of petrochemical origin, whose negative effects are particularly evident in relation to these aspects. They are instead not attributable to the C16-C18 triglycerides.

TABLE
Comparison between the reference formulation and that of Example 1

| Impact Category | Unit | Total | Reference | Example 1 | Improvement |
|---|---|---|---|---|---|
| Climate change Human | DALY | 1.13E−06 | 100% | 93% | 7% |
| Health | | | | | |
| Ozone depletion | DALY | 4.63E−10 | 100% | 83% | 17% |
| Human toxicity | DALY | 1.65E−07 | 88% | 100% | −12% |
| Photochemical oxidant | DALY | 1.67E−10 | 100% | 95% | 5% |
| formation | | | | | |
| Particulate matter | DALY | 4.51E−07 | 100% | 93% | 7% |
| formation | | | | | |
| Ionising radiation | DALY | 2.12E−09 | 100% | 96% | 4% |
| Climate change | species.yr | 6.43E−09 | 100% | 93% | 7% |
| Ecosystems | | | | | |
| Terrestrial acidification | species.yr | 2.52E−11 | 100% | 96% | 4% |
| Freshwater eutrophication | species.yr | 8.35E−12 | 100% | 90% | 10% |
| Terrestrial ecotoxicity | species.yr | 9.5E−10 | 100% | 81% | 19% |
| Freshwater ecotoxicity | species.yr | 1.28E−12 | 100% | 93% | 7% |
| Marine ecotoxicity | species.yr | 3.3E−15 | 100% | 99.6% | 0% |
| Agricultural land | species.yr | 2.48E−09 | 100% | 51% | 49% |
| occupation | | | | | |
| Urban land occupation | species.yr | 9.55E−11 | 100% | 88% | 12% |
| Natural land transformation | species.yr | 1.98E−07 | 100% | 81% | 19% |
| Metal depletion | $ | 0.002413 | 100% | 85% | 15% |
| Fossil depletion | $ | 4.933397 | 99% | 100% | −1% |

2. Results of the evaluation with the Eco Indicator 99 method

For the reference formulation and that of Example 1, the analysis carried out using the Eco Indicator method has provided the results shown in the graphs in FIGS. 3 and 4, respectively. Results are very similar to those obtained with the ReCiPe method.

C16-C18 triglycerides show a very low impact (max 4%), particularly by comparing it to that of the caprylic/capric triglyceride they replaced.

### BRIEF DESCRIPTION OF THE FIGURES

FIG. 1: Gelling effect of C16-C18 triglycerides (trade name “Olifeel Pearls”) at 2.5% in a variety of oils for cosmetic use

FIG. 2: Gelling effect of C16-C18 triglycerides (trade name “Olifeel Pearls”) at 5% in a variety of oils for cosmetic use

FIG. 3: Gelling effect of C16-C18 triglycerides (trade name “Olifeel Pearls”) at 10% in a variety of oils for cosmetic use FIG. 4: Gelling effect of C16-C18 triglycerides (trade name “Olifeel Pearls”) at 15% in a variety of oils for cosmetic use

FIG. 5: Gelling effect of C16-C18 triglycerides (trade name “Olifeel Pearls”) in a variety of oils for cosmetic use, summary (2,5, 5, 10, 15%)

FIG. 6: Characterisation of the reference formulation with the ReCiPe method

FIG. 7: Characterisation of the formulation of Example 1 with the ReCiPe method

FIG. 8: Characterisation of the reference formulation with the Eco Indicator 99 method

FIG. 9: Characterisation of the formulation of Example 1 with the Eco Indicator 99 method

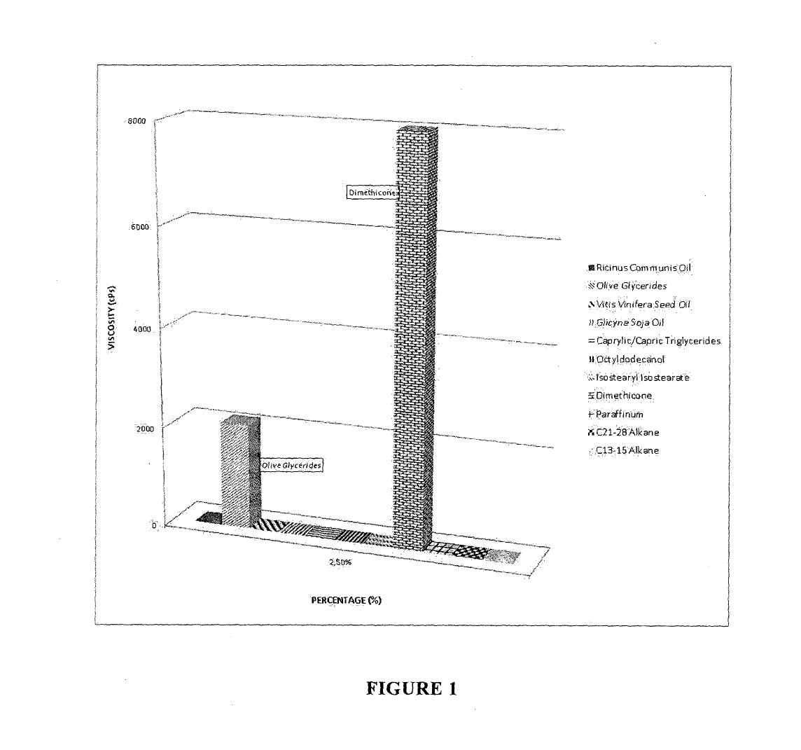

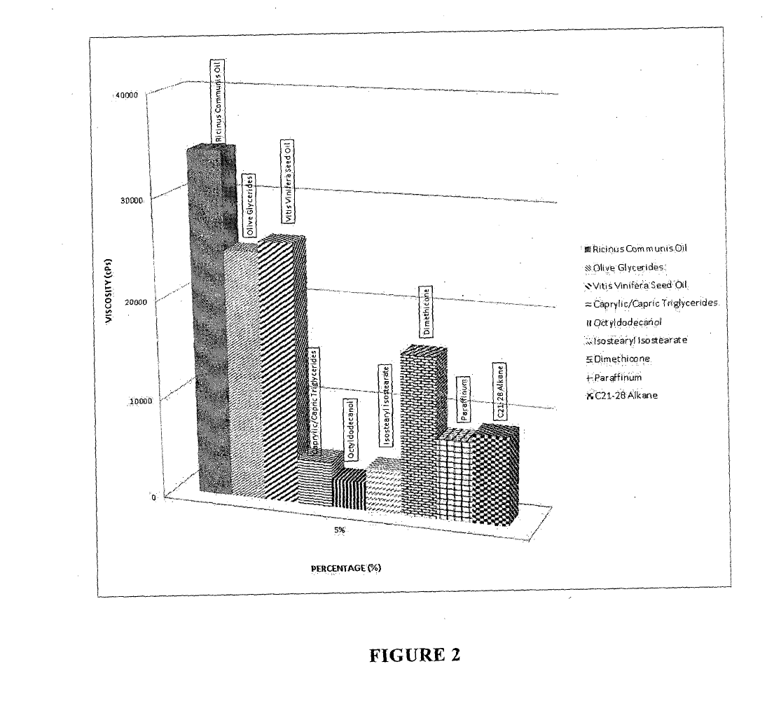

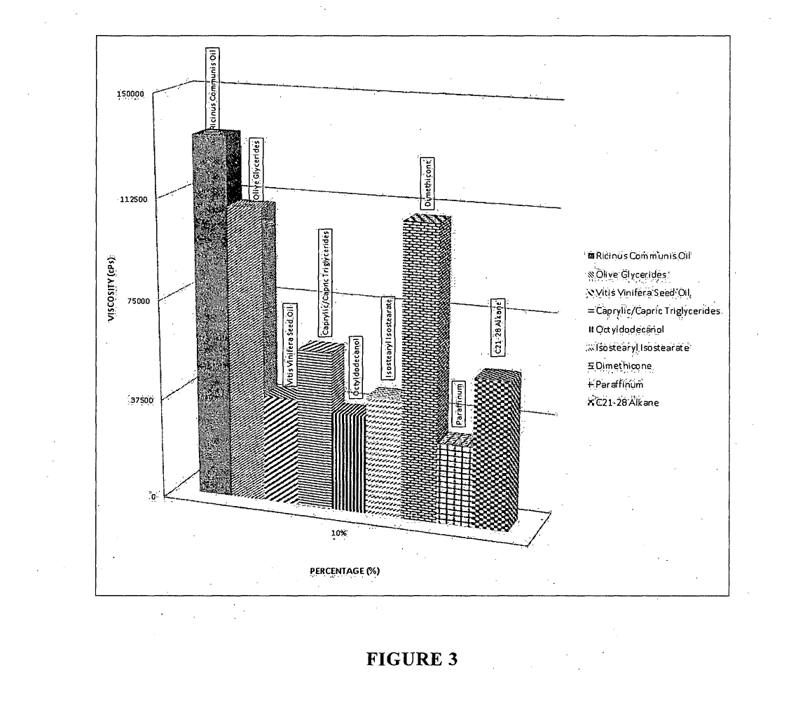

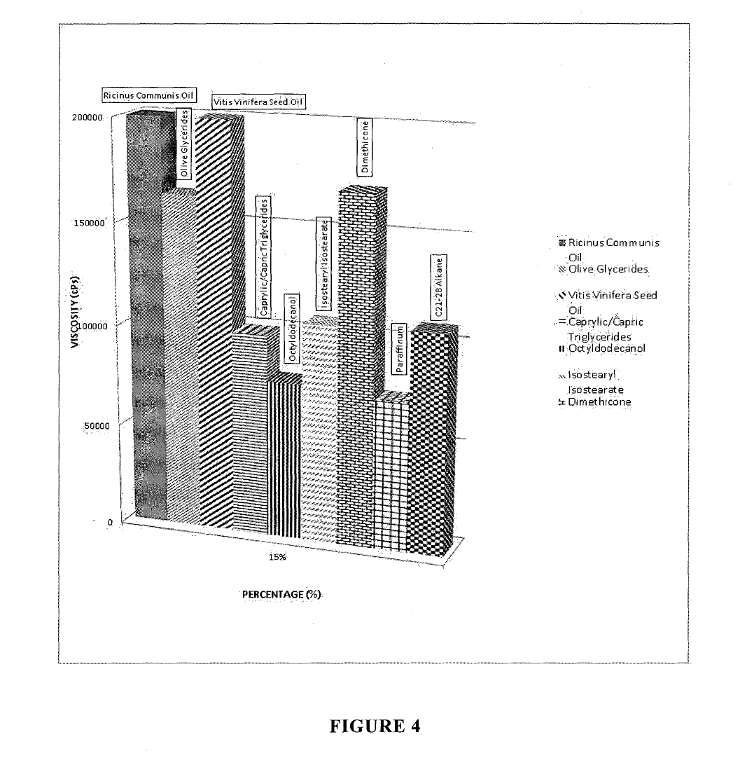

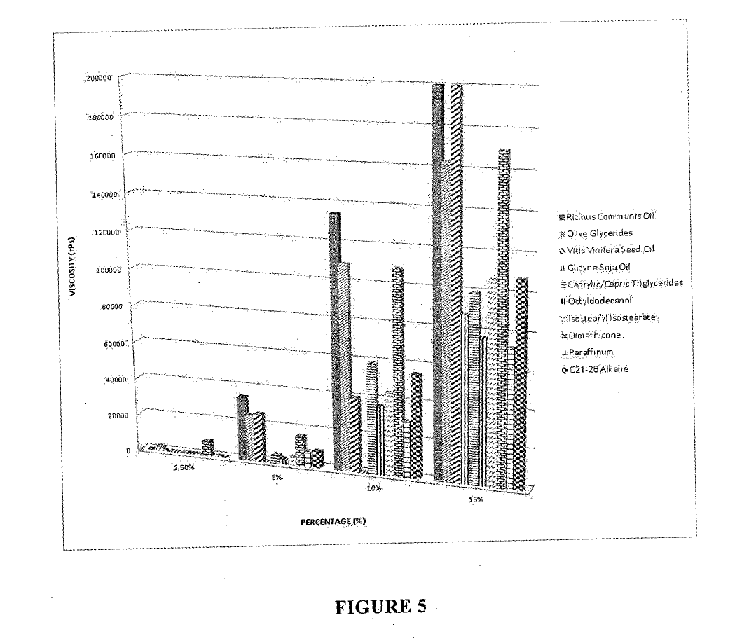

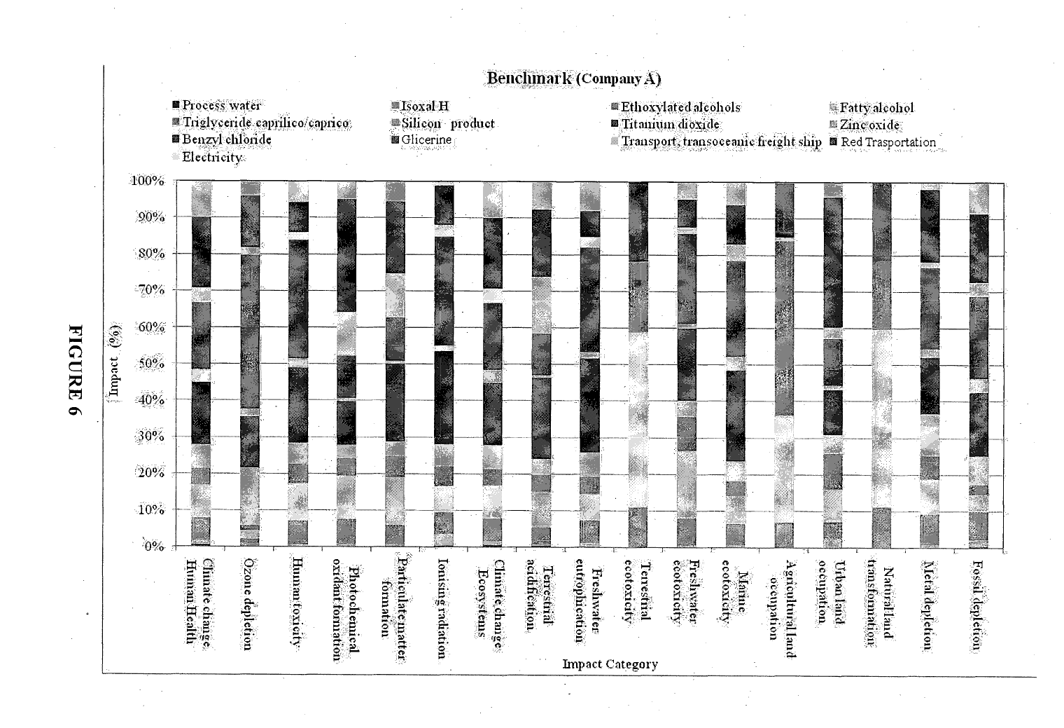

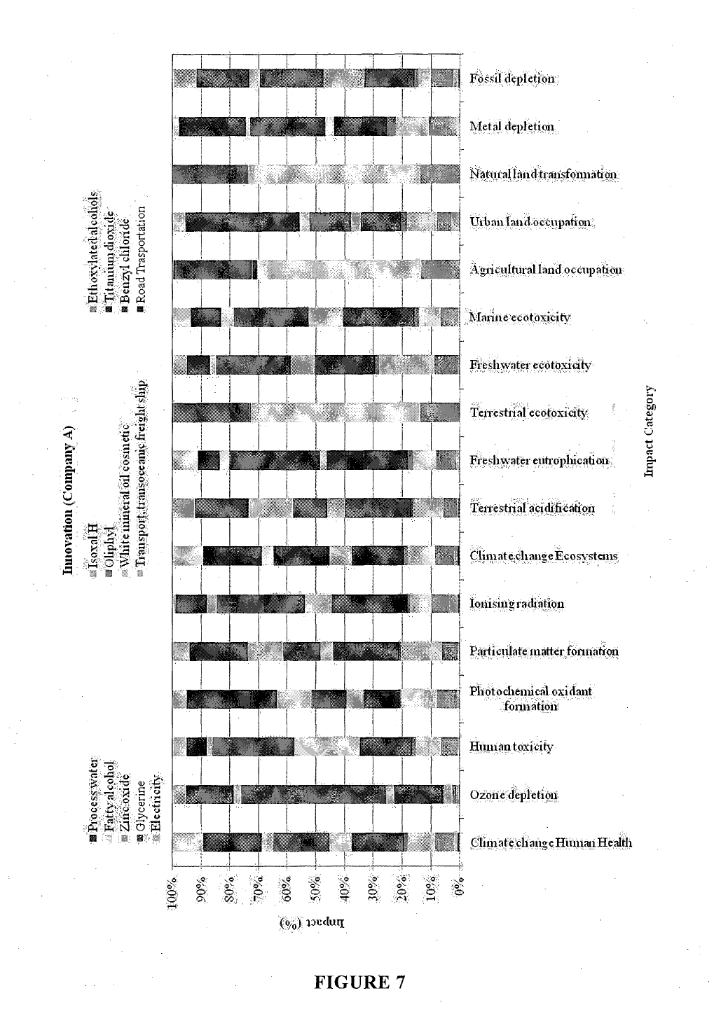

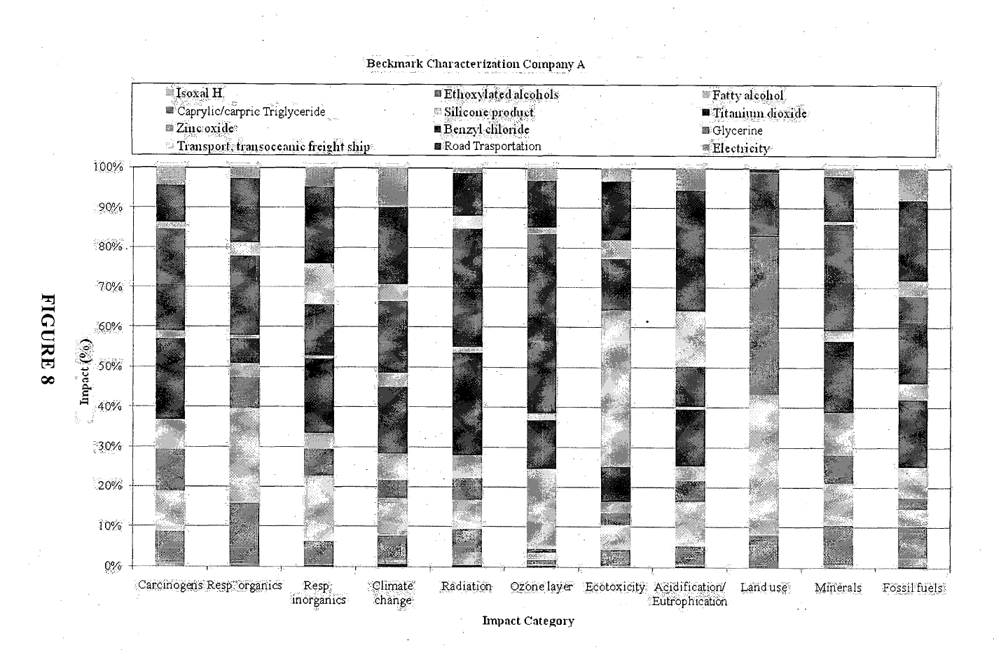

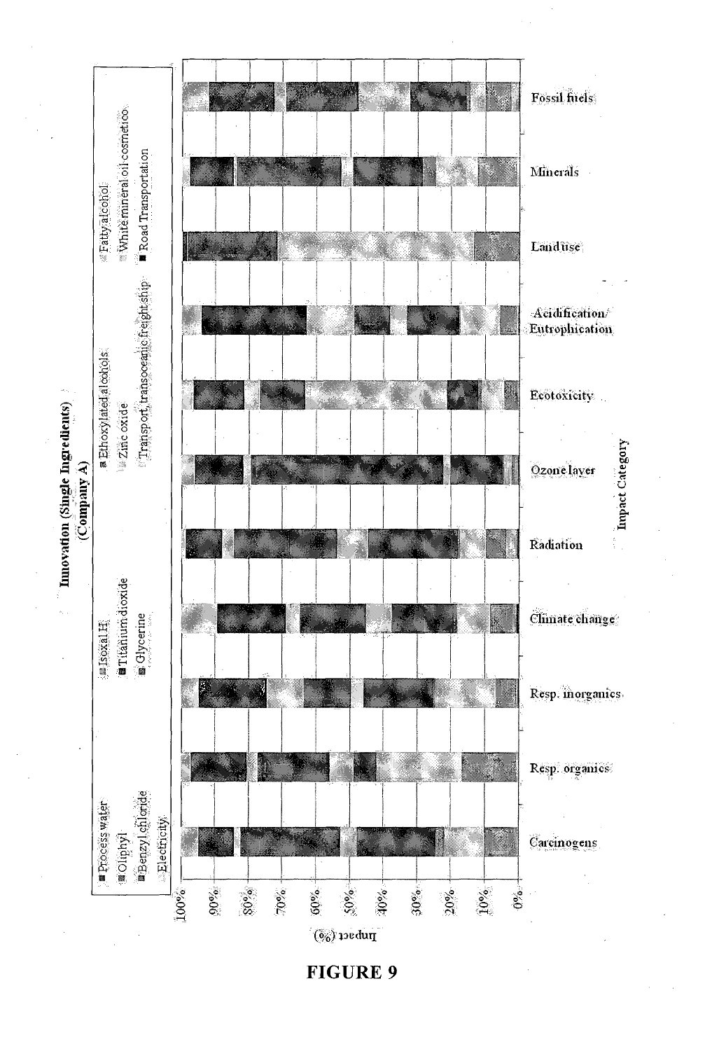

1. Composition for cosmetic use containing a mixture formed by triglycerides with high content in esters of fatty acids with saturated C₁₆-C₁₈ chain, and an unsaponifiable fraction, said mixture being obtained from deodorization distillates from the industrial processing of olive oil through the following procedure:
a) dispersing the deodorization distillate in cold water through the use of a surfactant and an electrolyte (adjuvants of crystallization of the fraction of saturated fatty acids);
b) after being left a few hours at ambient temperature, subjecting the dispersion to rapid “retempering”, then separating by centrifugation, obtaining a solid fraction, rich in free palmitic and stearic fatty acids, and a liquid fraction, consisting mainly of oleic and linoleic acids, and the entire unsaponifiable fraction of;
c) subjecting the solid fraction, rich in C₁₆-C₁₈ saturated acids to esterification by adding glycerol of vegetable origin, in excess of 10% compared to the stoichiometric ratio; under vacuum, heating from 165° C. up to 230° C. under forced circulation, and monitoring the production of water and the residual acidity, until when there is no more production of reaction water and the free acidity reaches a value of approx. 2-3%;
d) neutralizing the excess acidity by adding caustic soda at 90° C. to obtain a soapy mixture, washing the soapy mixture with demineralized water, and drying the mixture under vacuum;
e) optionally, further subjecting the fraction to bleaching and deodorization;
and, independently from steps c)-e):
f) subjecting the liquid fraction referred to in point b), rich in unsaturated triglycerides and unsaponifiables, to saponification with potassium hydroxide to remove the triglyceride fraction;
g) optionally, purifying the unsaponifiable fraction, consisting predominantly of squalene (approx. 70%), sterols and tocopherols by distillation in high vacuum (1 mbar residual pressure) from 100 to 250° C.; and
h) joining the fraction from step f) or g) to the product obtained in step d) or e).

2. Composition according to claim 1, wherein the content of triglyceride esters of fatty acids with C₁₆-C₁₈ saturated chain ranges from 90 to 98% of the total weight of the mixture.

3. Composition according to claim 1, wherein the weight ratio of saturated fatty acids C₁₆-C₁₈ in said mixture of triglycerides ranges from 3.5:1 to 6:1.

4. Composition according to claim 1, where by-products of the processing of palm, coconut, sunflower and corn oils are added in different proportions to the initial deodorization distillates of the industrial processing of the olive oil.

5. Composition according to claims 1, in the form of a bead (“pearl”) obtained through the following procedure:
a) heating the mixture, solid at room temperature, to a temperature of 90° C. in a melting vessel equipped with mechanical stirring;
b) after the mixture is completely melted, lowering the temperature to 80-85° C.;
c) casting the mixture from the melting vessel through a tap, on a percolator with rotating toothed shaft, and from here dripping said mixture on a refrigerant rotating band of a length of 18-20 m and at temperature of 8° C.;
d) collecting, at the end of the rotating band, the thus obtained solidified pearls in the desired packaging;
e) heating said mixture with stirring at fuser mechanical shovels until complete fusion.

6. Composition according to claim 1, further comprising a mixture of tri- and di-glycerides derived from olive oil processing.

7. Composition according to claim 6, wherein said tri- and di-glycerides are in a weight ratio of 75:25.

8. Composition according to claim 1, wherein the weight ratio of the mixture and said further mixture of tri- and di-glycerides is at least 1:4.

9. Composition according to claim 1, further comprising olive oil, preferably extra-virgin olive oil.

10. Composition according to claim 9, wherein the weight ratio of the mixture according to claim 1 and olive oil is equal to 3:7.

11. Cosmetic formulation containing a composition according to claim 1.

12. Formulation according to claim 11, in the form of cream, gel, ointment, cosmetic oil, massage oil, body/hand/face lotion, water/oil or oil/water emulsion, detergent, shampoo, hair lotion, bath gel and foam, shower gel and foam, liquid soap, stick, make-up products.

13. A method of making emulsions for cosmetic applications comprising adding an effective amount of the composition of claim 1 as a thickener of the lipophilic phases.

14. The composition of claim 1, wherein the glycerol of step c) is obtained by purification of olive oil.
